(19) United States
(12) Patent Application Publication
(10) Pub. No.: US 2025/0367042 A1
(43) Pub. Date: Dec. 4, 2025

(54) DIAPER WITH COLLECTION POUCH

(71) Applicant: c/o EASYDAY HEALTH PRODUCTS INC., Blainville (CA)

(72) Inventors: Cristian STEFU, Blainville (CA); Ioana Mihaela HIDISAN, Blainville (CA); Regent RACINE, Blainville (CA)

(21) Appl. No.: 19/206,403

(22) Filed: May 13, 2025

Related U.S. Application Data
(60) Provisional application No. 63/646,312, filed on May 13, 2024.

(51) Int. Cl. A61F 13/495; A61F 13/511; A61F 13/84

(52) U.S. Cl. CPC A61F 13/495; A61F 13/51104; A61F 13/8405; A61F 2013/4953; A61F 2013/8414

(57) ABSTRACT

A diaper comprises a backing layer defining an outer side and an opposite inner side. An absorbent core defines a top surface and an opposite underside surface overlying the inner side of the backing layer. At least a substantial portion of the underside surface is unconnected to at least a substantial portion the inner side. An opening is formed along a length of the absorbent core extending from the top surface to the underside surface. A peripheral border structure surrounds the backing sheet and the absorbent core and is integrated therewith. The peripheral border structure and the unconnected portions of the underside surface and the inner side form a pouch therebetween accessible via the opening. When the diaper is worn by a user, the opening is positioned about the anus of a user for providing for fecal matter to enter the pouch during defecation.

### CROSS-REFERENCE TO RELATED APPLICATIONS

The present application claims priority on U.S. Provisional Patent Application Ser. No. 63/646,312 filed on May 13, 2024, and incorporated by reference herein in its entirety.

### TECHNICAL FIELD

The present disclosure generally relates to diapers. More particularly but not exclusively the present disclosure relates to a diaper with a collection pouch.

### BACKGROUND

Diapers include both disposable baby diapers and adult diapers. Diapers can be necessary for adults with various conditions, such as incontinence, mobility impairment, severe diarrhea, or dementia. A diaper is a type of underwear that allows the wearer to defecate or urinate without the use of a toilet, by absorbing or containing waste products to prevent soiling of outer clothing or the external environment. When diapers become soiled, they require changing, generally by a second person such as a parent or a caregiver. Diapers have a facing layer to be brought into contact with the wearer's skin, an absorbent panel adjacent thereto, and a water-impervious or a water repellant outer layer. Following defecation, prolonged contact of feces with the wearer's skin may have adverse health consequences such as rashes or infections. For caregivers, changing the soiled diapers of adult patients poses many practical challenges as the task is usually extremely messy with fecal matter spilling out of the diapers and onto the sheets of the patient's bed. The ordeal worsens with patients suffering with sever diarrhea and other digestive issues.

A prior diaper collection pouch has been described in International Patent Application No. PCT/CA2020/000028 filed on Mar. 16, 2020 as well as in International Patent Application No. PCT/CA2023/050592 filed on May 2, 2023, both of which are incorporated herein by reference in their entirety. The foregoing documents teach a diaper that comprises a bottom layer and a top layer mounted thereto. The bottom layer defines a top surface and an opposite outer surface and comprises an absorbent core therebetween. The top layer is mounted to the bottom layer about a peripheral border thereof. The top layer defines a top layer area delimited by the peripheral border overlying the top surface and being unconnected to the bottom layer. The top layer area, the top surface and the peripheral border define a pouch therebetween. The top layer area defines a body contacting surface and an opening for providing access to the pouch. When the diaper is worn by the user, the opening is positioned about the anus of a user for providing for fecal matter to enter the pouch during defecation.

### Objects

An object of the present disclosure is to provide a diaper with a collection pouch.

### SUMMARY

In accordance with an aspect of the present disclosure, there is provided a diaper comprising: a backing layer defining an outer side thereof and an opposite inner side thereof; an absorbent core defining a top surface thereof and an opposite underside surface overlying the inner side of the backing layer, at least a substantial portion of the underside surface being unconnected to at least a substantial portion the inner side of the backing layer, an opening being formed along a given length of the absorbent core and extending from the top surface to the underside surface; and a peripheral border structure surrounding the backing sheet and the absorbent core and being integrated therewith, the peripheral border structure and the unconnected substantial portions of the underside surface and the inner side forming a pouch therebetween accessible via the opening, wherein when the diaper is worn by a user, the opening is positioned about the anus of a user for providing for fecal matter to enter the pouch during defecation.

In an embodiment, the opening is provided by a cut line being formed along the given length of the absorbent core and extending from the top surface to the underside surface

In an embodiment, the diaper further comprises a top layer overlying the top surface of the absorbent core for being in contact with the user when worn.

In an embodiment, the top layer defines a tear line along the cut line for providing the opening therewith.

In an embodiment, the cut line is selectively openable about a selected length thereof in order to define the opening.

In an embodiment, the tear line is selectively openable about a selected length thereof in order to define the opening.

In an embodiment, the opening is positioned along the intergluteal cleft of the user when worn.

In an embodiment, the diaper further comprises a spacer positioned within the pouch for providing a space between the underside surface and the inner side.

In an embodiment, the spacer comprises an insert comprised of cuts and folds that when pulled increases in height thereby lifting the underside surface away from the inner side to provide the space.

In an embodiment, the insert when increased in height forms cavities for allowing passage to the fecal matter.

In an embodiment, the spacer comprises anti-odorant and/or anti-microbial agents.

In an embodiment, the diaper further comprises anti-odorant and/or anti-microbial agents within the pouch.

In an embodiment, the cut line is formed by a plurality of separate linear cuts along the length of the absorbent core, separating the absorbent core into two adjacent sides about the cut line, wherein each of the two sides define a plurality of spaced apart additional cut lines intersecting the linear cuts to form collapsible finger elements therewith that are collapsible into the opening when engaged by the fecal matter thereby guiding the fecal matter into the pouch.

In an embodiment, the top surface of the absorbent core forms channels for guiding the fecal matter to the opening.

In an embodiment, the underside surface and the inner side comprise slippery surface material.

In an embodiment, the inner side comprises absorption material.

In an embodiment, the absorbent core comprises an upper part thereof and a lower part thereof, wherein the upper part therefor provides for greater absorption of liquid than the lower part.

In an embodiment, the absorbent core comprises a front portion thereof and a rear portion thereof, wherein the front portion provides for greater absorption of liquid than the rear portion.

In an embodiment, the underside surface comprises curvature line formations providing for guiding fecal matter flow radially away from the opening.

Other objects, advantages and features of the present disclosure will become more apparent upon reading of the following non-restrictive description of illustrative embodiments thereof, given by way of example only with reference to the accompanying drawings.

### BRIEF DESCRIPTION OF THE DRAWINGS

The following drawings form part of the present specification and present disclosure. In the appended drawings:

FIG. 1 is a top plan view of the inner side of the diaper in accordance with a non-restrictive illustrative embodiment of the present disclosure;

FIG. 2 is a bottom view of the outer side of the diaper of FIG. 1 in accordance with a non-restrictive illustrative embodiment of the present disclosure;

FIG. 3 is a schematic sectional view along a length of the diaper in a flat position in accordance with a non-restrictive illustrative embodiment of the present disclosure;

FIG. 4 is a schematic sectional view along a length of the diaper in a curved position in accordance with a non-restrictive illustrative embodiment of the present disclosure;

FIG. 5 is a schematic section view of the absorption core of the diaper in accordance with a non-restrictive illustrative embodiment of the present disclosure;

FIG. 6 is a top view of the inner side of the diaper without a top layer covering the absorption core in accordance with a non-restrictive illustrative embodiment of the present disclosure;

FIG. 7 is a bottom view of the rear side of the absorption core of the diaper in accordance with a non-restrictive illustrative embodiment of the present disclosure;

FIG. 8 is an exploded perspective view of the diaper in accordance with a non-restrictive illustrative embodiment of the present disclosure;

FIG. 9 is another exploded perspective view of the diaper in accordance with a non-restrictive illustrative embodiment of the present disclosure;

FIG. 10 is a schematic close up top view of the cut line of the absorption core of the diaper in accordance with a non-restrictive illustrative embodiment of the present disclosure;

FIG. 11 is another schematic close up top view of the cut line of the absorption core of the diaper in accordance with a non-restrictive illustrative embodiment of the present disclosure;

FIG. 12 shows perspective views of the insert spacer of the diaper from the flat (I) to lifted (IV) positions and the transitional (II, III) positions therebetween diaper in accordance with a non-restrictive illustrative embodiment of the present disclosure;

FIG. 13 shows an insert spacer being positioned on the inner side of the backing sheet of the diaper in accordance with a non-restrictive illustrative embodiment of the present disclosure; and

FIG. 14 is an exploded perspective view of the diaper in accordance with a non-restrictive illustrative embodiment of the present disclosure.

### DETAILED DESCRIPTION

Generally stated, there is provided a diaper comprising a backing layer defining an outer side thereof and an opposite inner side thereof. An absorbent core defining a top surface thereof and an opposite underside surface overlies the inner side of the backing layer. At least a substantial portion of the underside surface is unconnected to at least a substantial portion the inner side surface of the backing layer. A cut line is formed along a length of the absorbent core and extends from the top surface to the underside surface to provide an opening. A peripheral border structure surrounds the backing sheet and the absorbent core and is integrated therewith. The peripheral border structure and the unconnected substantial portions of the underside surface and the inner side form a pouch therebetween accessible via the opening. When the diaper is worn by a user, the opening is positioned about the anus of a user for providing for fecal matter to enter the pouch during defecation.

With reference to the Figures, non-restrictive illustrative embodiment of the present disclosure will now be described.

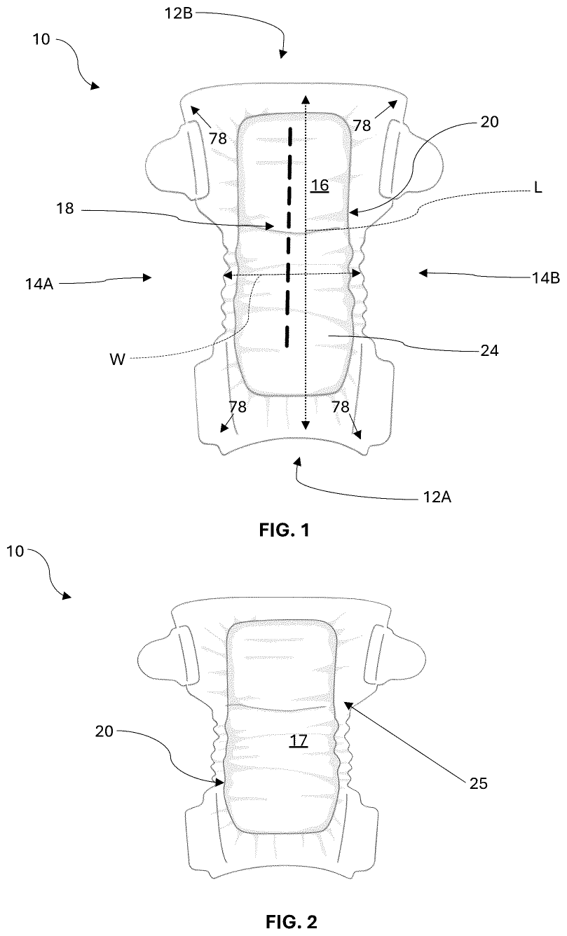

FIGS. 1 and 2 show a diaper 10 in accordance with an illustrative embodiment of the present disclosure with front and rear ends, 12A and 12B, respectively, and opposite lateral sides 14A and 14B.

The diaper 10 includes an inner side 16 and an opposite outer side 17. The inner side 16 is in direct contact with the skin of the user. The inner side 16 includes a tear or cut line 18 which when opened provides for access to an internal pouch. The diaper 10 includes a central absorbing part 20 including the cut line 18. The central absorbing part 20 absorbs and stores user discharge such as urine and feces.

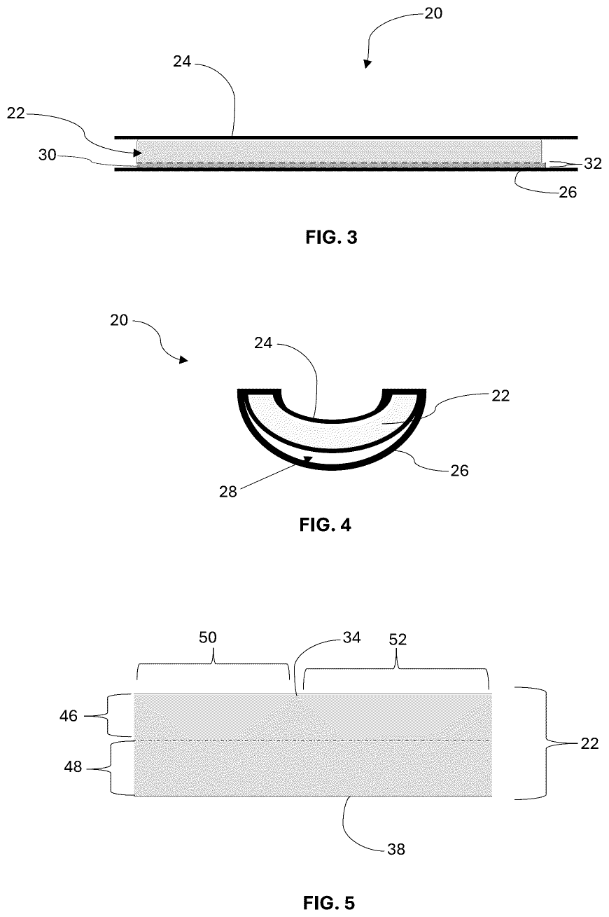

As shown in FIGS. 3 and 4, the central absorbing part 20 comprises an absorption core 22.

A top layer comprising a top sheet 24 overlies the absorption core 22 and is in direct contact with the user and forms part of the inner side 16. The top sheet 24 is laminated to the absorption core 22 such that there is no space therebetween. The absorption core 22 overlies a backing layer comprising a backing sheet 26 that is laminated at its edges to the top sheet 24 for fittingly enveloping the absorption core 22 therebetween.

The diaper 10 defines a peripheral boundary 25 structure that surrounds and is integral to the absorbent core 22 and the backing layer as well as the top layer.

The top sheet 24 is comfortable for the wearer and provides for absorbing discharge. The backing sheet 26 is water-resistant to prevent liquids from passing through the diaper 10 and leaking onto the clothing or surrounding environment. In an embodiment, the backing sheet 26 allows water vapor and air (but not liquids) to pass through, reducing moisture and keeping the skin drier.

The absorption core 22 or at least a substantial portion thereof overlies the outer backing sheet 26 but is not connected thereto. As such, and as shown in FIG. 4, a pocket or pouch 28 is formed between the absorption core 22 and the backing sheet 26.

The absorption core 22 is cut along the tear line 18 to provide access to the pouch 28 thereunder.

Turning back to FIG. 3, a spacer 30 can be provided between the absorption core 22 and the backing sheet 26 to provide a space 32 therebetween which assists in forming the size of the pouch 30 by providing a minimal distance (i.e. space 32) between the absorption core 22 and the backing sheet 26.

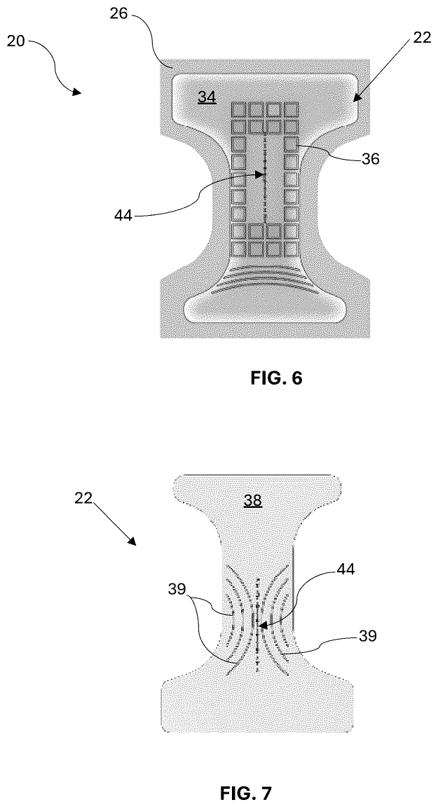

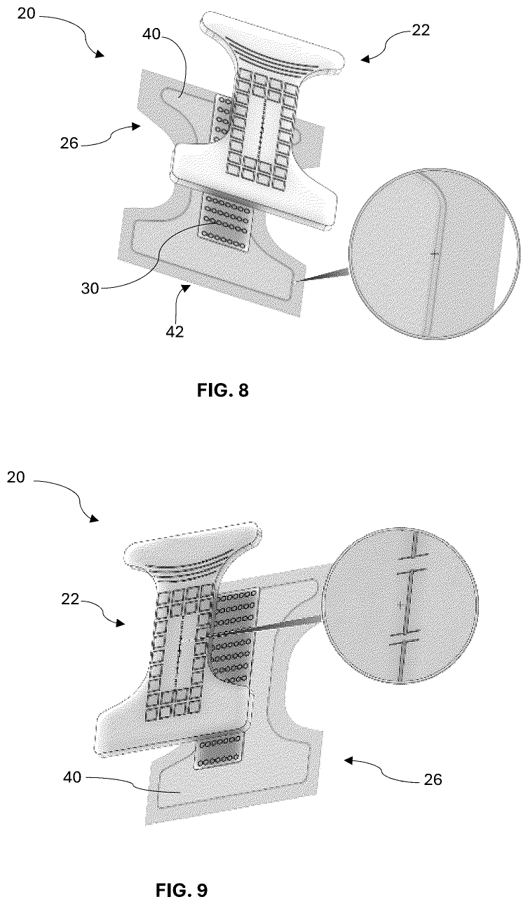

With reference to FIGS. 6 and 7, the absorption core 22 defines a top surface 34 with absorption padding 36 and an underside surface 38. A cut line 44 is cut through the absorption core 22 from the top surface 34 to the underside surface 38 providing an opening into the pouch 28. The top surface 34 is covered by the top sheet 24 and the underside surface 38 overlies the backing sheet 26 without connection therebetween as previously described.

The curvature line formations 39 in the underside surface 38 shown in FIG. 7 provide for guiding fecal matter flow radially away from the center cut line 44.

With reference to FIGS. 8 and 7, the absorption core 22 is positioned on the inner side 40 of the backing sheet 26 which together with the underside 38 of the absorption core 22 define the pocket 28. A spacer 30 is positioned in the inner side 40 for providing the space 32 between surfaces 30 and 38. The backing sheet 26 defines an opposite outer side 42 which forms part of the outer side 17.

With reference to FIG. 5, the absorption core 22 defines a top part 46 and a bottom part 48. The top part 46 comprises the top surface 34 and the bottom part 48 comprises the underside surface 38. In an embodiment, the top part 46 provides for greater absorption of liquid discharge while the bottom part 48 provides for greater absorption of fecal matter. In an embodiment, the front portion 50 of the top part 46 has an increased absorption characteristics to provide for greater absorption of liquids as it is near the urethra. Indeed, the rear portion 52 of the top part 46 can be less absorptive in order for liquid to be drawn to the front portion 50.

The cut line 44 is positioned along the center line of the intergluteal cleft.

In an embodiment, the cut line 44 and the tear line 18 are one and the same and as such can be referred to as a tear line providing small cuts along the line 18 allowing a user to selectively tear the central absorption part 20 (along with the absorption core 22) along the line 18 at a desired length and position thereof to conveniently provide a sufficient opening positioned about the anus.

In an embodiment, instead of tear lines and cut lines, lines 18 and 44 are one and the same and define a pre-formed opening without the need of cuts and tearing.

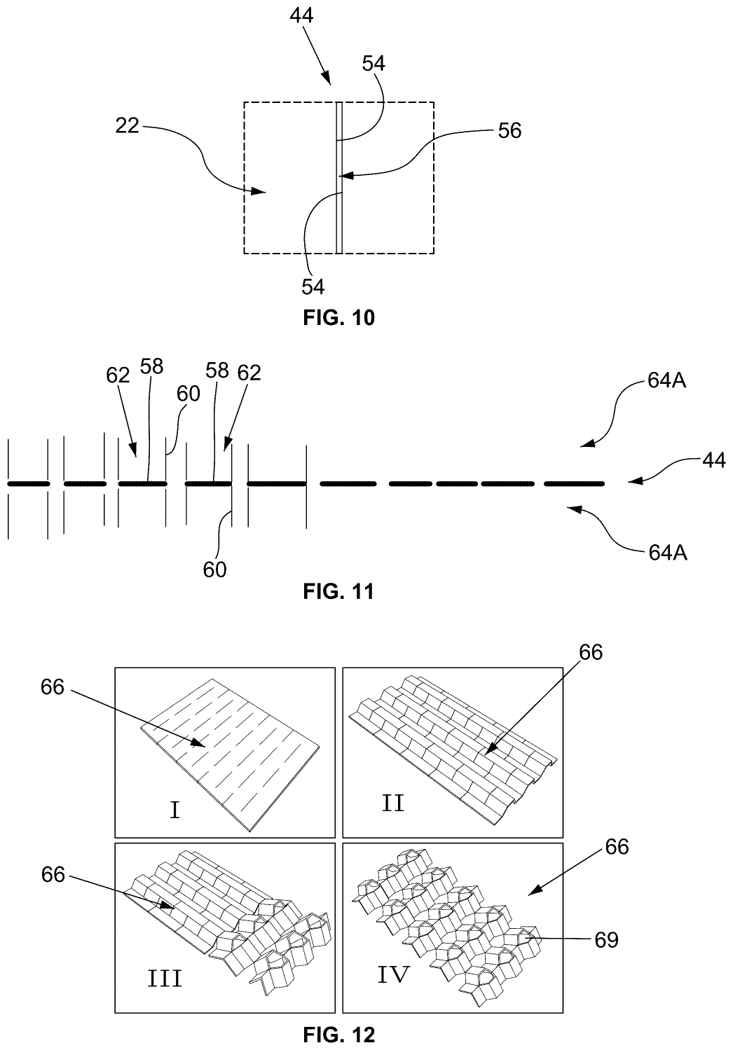

As shown in FIG. 10, the cut 44 forms two opposite and interfacing surfaces 54 defining the opening 56 therebetween and providing for absorption of liquid discharge matter flowing thereon. These inner side interfacing surface 54 have a thickness corresponding to the thickness of the absorption core 22.

In an embodiment, the top sheet 24 laminated to the absorption core 22 do not include cut lines or tear lines but rather have an opening 56 preformed therethrough. In an embodiment the opening defines a preformed gap as it has slightly spaced apart interfacing surfaces 54 as shown in FIG. 10.

Fecal matter flows into the pouch 28 via the opening 56 and liquid therein is absorbed via the underside surface 38 bottom part 48 of the absorption core 22 with the rest of the substantially solid matter being stored in the pouch 28. The backing sheet 26 prevents liquid flow therefrom and in this way allows for absorption via the underside surface 38. As such, fecal matter is stored away from the skin of the user. Thus, the inner side surfaces 54 and the underside surface 38 of the absorption core act as sponge surfaces for absorbing liquid in the fecal matter.

Turning to FIG. 11, the cut line 44 is made of series of small linear cuts 58 along the length L of the inner side 16 (see FIG. 1), the absorption core 22 also includes cuts 60 along the width W of the inner side 16 (see FIG. 1) which here are shown to be generally perpendicular to the linear cuts 58 thereby forming rectangular fingers 62. Indeed, the cuts 60 may be angled to provide triangular fingers for example.

The fingers 62 at each side 64A and 64B of the cut 44 collapse inwardly into the opening 56 formed by the cut line 44 when engaged by fecal matter guiding the feces inwardly into the pouch 28.

The top sheet 24 can include adhesive material or material that adheres to the skin of the user in order to bring the cut line 44 closer to the intergluteal cleft and the opening 56 near the anus. Thus, the top sheet 24 is sealed to the skin providing for the fecal matter to be directed into the opening 56 as the path of least resistance. This is aided by the collapsing sides 64A and 64B.

Furthermore, the underside surface 38 and the inner side 40 forming the pouch 28 comprises a slippery surface to allow the feces to more easily slide therein. In embodiment, this slippery surface comprises small channels for guiding the feces in the desired direction. In an embodiment, the inner side 40 of a portion or portions thereof comprise absorbent material.

In an embodiment, the pouch 28 may include anti-odorant and anti-microbial agents.

In an embodiment and with reference to FIG. 12, the spacer 30 comprises an insert 66 of one or more layers of a thin sheet with purposely designed cuts and folds such as Kirigami paper which is flat but when pulled lifts up to reveal three dimensional structures as shown in positions I, II, III and IV.

The purpose of insert 66 is to provide the lift between the surfaces 38 and 40 defining the pouch 28 thus creating the space for the flow of the feces. In an embodiment, when initially positioned during manufacturing the insert 66 is completely flat (position I) and as thin as a regular paper (0.05-0.10 mm). When the user wears the diaper, insert 66 is pulled and gradually lifted (positions II, III and IV). The lifted insert 66 is designed to support the weight of the user and to provide openings 69 which provide flow passages with unidirectional flow properties. Since the insert 66 is not in contact with the user it can contain various anti-odorant and anti-microbial agents.

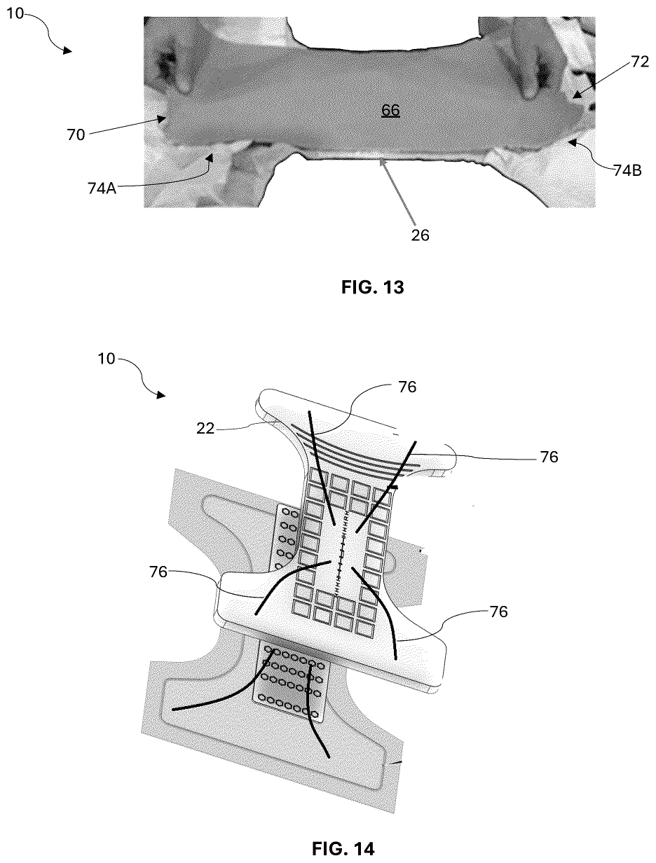

In an embodiment and with reference to FIG. 13, when the diaper 10 is being assembled, the front and rear ends 70 and 72 of the spacer insert 66 are respectively connected to the front and rear inner fold portions 74A and 74B of the backing layer 26 so that when the diaper 10 is unfolded for being worn, the front and rear inner fold portions 74A and 74B respectively and oppositely pull front and rear ends 70 and 72 forwardly and rearwardly thereby stretching the insert 66 causing it to move from the flat position to the lifted position as explained above.

In an embodiment, strings are attached to the front and rear ends 70 and 72 of the spacer insert 66 and extends outwardly of the diaper 10 from the front and rear ends 12A, 12B, respectively thereof to be pulled by the user to stretch the insert 66 causing it to move from the flat position to the lifted position as explained above.

In an embodiment, a thin cord below the absorption core 22 is provided and attached in the rear of the diaper 10 and extending to the front of the diaper. When the diaper is worn by the user the caregiver pulls this cord such that the sides 64A and 64B of the core 22 about the cut line 44 slightly enter the internuclear cleft.

In an embodiment, top surface 34 of the absorption core 22 and the top sheet 24 overlying this top surface are formed with wave like channels allowing urine and feces to distance discharge from the skin of the user and to guide the fecal matter towards the opening 56 into the pouch 28.

The waves form peaks and valleys, the peaks will collapse under the weight of the user, but the valleys will remain in place to form channels for fitting discharge therein for absorption thereof and for guiding the feces towards the opening 56.

When removing the diaper 10, the adhesive material on the inner 16 allows the caregiver to fold and seal the diaper 10 for safer disposal.

Separating the absorption core into left and right parts provides for each part to take the shape of a respective buttock and more securely be fitted to the wearer with the cut line running along the intergluteal cleft.

Turning to FIG. 14, the absorption core 22 comprises body shape adaptation lines 76 to better fit on the human body. These lines 76 provide a better gap between the backing layer 26 and the absorption core 22. Indeed, the corner sections 78 (see FIG. 1) of the inner diaper side when all its parts are assembled provide seams which are gripping or anchoring points to snuggly fit the diaper 10 on the human body.

The diaper 10 can be made of a variety of suitable materials and designed in a variety of suitable configurations within the context of the present disclosure.

The various features described herein can be combined in a variety of ways within the context of the present disclosure so as to provide still other embodiments. As such, the embodiments are not mutually exclusive. Elements of the embodiments and examples discussed can be combined within the context of the disclosure as those having skill in the art will readily appreciate. Moreover, the embodiments discussed herein need not include all of the features and elements illustrated and/or described and thus partial combinations of features can also be contemplated. Furthermore, embodiments with less features than those described can also be contemplated.

It is to be understood that the present disclosure is not limited in its application to the details of construction and parts illustrated in the accompanying drawings and described hereinabove. The disclosure is capable of other embodiments and of being practiced in various ways. It is also to be understood that the phraseology or terminology used herein is for the purpose of description and not limitation. Hence, although the present disclosure has been provided hereinabove by way of non-restrictive illustrative embodiments thereof, it can be modified, without departing from the scope, spirit and nature thereof and of the appended claims.

What is claimed is:

1. A diaper comprising:
a backing layer defining an outer side thereof and an opposite inner side thereof;
an absorbent core defining a top surface thereof and an opposite underside surface overlying the inner side of the backing layer, at least a substantial portion of the underside surface being unconnected to at least a substantial portion the inner side of the backing layer, an opening being formed along a given length of the absorbent core and extending from the top surface to the underside surface; and
a peripheral border structure surrounding the backing sheet and the absorbent core and being integrated therewith, the peripheral border structure and the unconnected substantial portions of the underside surface and the inner side forming a pouch therebetween accessible via the opening,
wherein when the diaper is worn by a user, the opening is positioned about the anus of a user for providing for fecal matter to enter the pouch during defecation.

2. A diaper according to claim 1, wherein the opening is provided by a cut line being formed along the given length of the absorbent core and extending from the top surface to the underside surface.

3. A diaper according to claim 1, further comprising a top layer overlying the top surface of the absorbent core for being in contact with the user when worn.

4. A diaper according to claim 2, further comprising a top layer overlying the top surface of the absorbent core for being in contact with the user when worn.

5. A diaper according to claim 4, wherein the top layer defines a tear line along the cut line for providing the opening therewith.

6. A diaper according to claim 2, wherein the cut line is selectively openable about a selected length thereof in order to define the opening.

7. A diaper according to claim 5, wherein the tear line is selectively openable about a selected length thereof in order to define the opening.

8. A diaper according to claim 1, wherein the opening is positioned along the intergluteal cleft of the user when worn.

9. A diaper according to claim 1, further comprising a spacer positioned within the pouch for providing a space between the underside surface and the inner side.

10. A diaper according to claim 9, wherein the spacer comprises an insert comprised of cuts and folds that when pulled increases in height thereby lifting the underside surface away from the inner side to provide the space.

11. A diaper according to claim 10, wherein the insert when increased in height forms cavities for allowing passage to the fecal matter.

12. A diaper according to claim 9, wherein the spacer comprises anti-odorant and/or anti-microbial agents.

13. A diaper according to claim 1, further comprising anti-odorant and/or anti-microbial agents within the pouch.

14. A diaper according to claim 2, wherein the cut line is formed by a plurality of separate linear cuts along the length of the absorbent core, separating the absorbent core into two adjacent sides about the cut line, wherein each of the two sides define a plurality of spaced apart additional cut lines intersecting the linear cuts to form collapsible finger elements therewith that are collapsible into the opening when engaged by the fecal matter thereby guiding the fecal matter into the pouch.

15. A diaper according to claim 1, wherein the top surface of the absorbent core forms channels for guiding the fecal matter to the opening.

16. A diaper according to claim 1, wherein the underside surface and the inner side comprise slippery surface material.

17. A diaper according to claim 1, wherein the inner side comprises absorption material.

18. A diaper according to claim 1, wherein the absorbent core comprises an upper part thereof and a lower part thereof, wherein the upper part therefor provides for greater absorption of liquid than the lower part.

19. A diaper according to claim 1, wherein the absorbent core comprises a front portion thereof and a rear portion thereof, wherein the front portion provides for greater absorption of liquid than the rear portion.

20. A diaper according to claim 1, wherein the underside surface comprises curvature line formations providing for guiding fecal matter flow radially away from the opening.
